(19) United States
(12) Patent Application Publication
(10) Pub. No.: US 2022/0117546 A1
(43) Pub. Date: Apr. 21, 2022

(54) WOUND MEASUREMENT

(71) Applicant: WoundMatrix, Inc., Chadds Ford, PA (US)

(72) Inventors: Jason Peter MURRAY, Chadds Ford, PA (US); Joshua LOWENTHAL, Glen Mills, PA (US); Ali ALDEEWAN, Newark, DE (US); Osama AL-MOOSAWI, Downington, PA (US)

(21) Appl. No.: 17/505,527

(22) Filed: Oct. 19, 2021

Related U.S. Application Data
(60) Provisional application No. 63/093,752, filed on Oct. 19, 2020.

(51) Int. Cl. A61B 5/00; G16H 30/00; G16H 50/20; G06T 7/13

(52) U.S. Cl. CPC A61B 5/445; G16H 30/00; G06T 2207/30088; G06T 7/13; G06T 2207/10024; G16H 50/20

(57) ABSTRACT

Systems and methods for detecting an edge-perimeter of a wound from a digital wound image are disclosed, which may include: receiving, as an initial sampling point, an input from a user of a point on the digital wound image located inside of the wound; determining an initial edge-perimeter of the wound based on the initial sampling point by determining an initial representative color space spectrum from the initial sampling point; determining an initial center point of the wound on the digital wound image based on the initial edge-perimeter; determining a final edge-perimeter of the wound by using the initial center point as a second sampling point and determining a second representative color space spectrum from the second sampling point; and displaying an overlay of the final edge-perimeter superimposed on the digital wound image.

### RELATED APPLICATIONS

This application claims the benefit of priority to U.S. provisional Application No. 63/093,752, filed Oct. 19, 2020, which is incorporated herein by reference in its entirety.

### TECHNICAL FIELD

This disclosure relates to medical, clinical, or therapeutic systems and methods for wound measurement and management. More particularly, embodiments of the present disclosure relate to inventive and unconventional systems and methods that capture digital images of wounds, evaluate the digital images to determine states of the wounds, and facilitate tracking the states of the wounds over time.

### BACKGROUND

For wound-care, it is well-recognized that healthcare practitioners' clinical and medical considerations and decisions based on test-result or other inputs must be well-informed, fact-based, competent, and justifiable. Accordingly, such inputs must be reasonably and reproducibly accurate, feasible for “practical use,” and consistent with results obtained by alternative measurement-methods or obtained as between wound measurements “performed by the same clinician or different clinicians.” Such inputs include, for example, a patient's overall health, their underlying medical conditions, types and causes of their wounds, clinical test-results, their wounds' healing rates (including the wounds' progressively changing measurements and appearances while healing), and other clinical observations or measurements.

While there have been attempts to design automatic or semi-automatic systems and methods for wound measurement, such systems or methods must incorporate controls or calculations for inherent variables and error in order to operate and report results consistently as a whole for an entire course or progressive series of image submissions. Thus, there exists among clinicians and other medical wound-care practitioners a long felt but still significantly unfulfilled need for systems and methods for accurate, objective, and consistent wound management.

### SUMMARY

One aspect of the present disclosure is directed to a computer-implemented method for detecting an edge-perimeter of a wound from a digital wound image. The method may comprise: receiving, as an initial sampling point, an input from a user of a point on the digital wound image located inside of the wound; determining an initial edge-perimeter of the wound based on the initial sampling point by determining an initial representative Red-Green-Blue (RGB) color space spectrum from the initial sampling point; determining an initial center point of the wound on the digital wound image based on the initial edge-perimeter; determining a final edge-perimeter of the wound by using the initial center point as a second sampling point and determining a second representative RGB color space spectrum from the second sampling point; and displaying an overlay of the final edge-perimeter superimposed on the digital wound image.

Another aspect of the present disclosure is directed to a computer-implemented system for detecting an edge-perimeter of a wound from a digital wound image. The system may comprise: at least one non-transitory computer-readable medium configured to store instructions; and at least one processor configured to execute the instructions to perform operations. The operations may comprise: receiving, as an initial sampling point, an input from a user of a point on the digital wound image located inside of the wound; determining an initial edge-perimeter of the wound based on the initial sampling point by determining an initial representative RGB color space spectrum from the initial sampling point; determining an initial center point of the wound on the digital wound image based on the initial edge-perimeter; determining a final edge-perimeter of the wound by using the initial center point as a second sampling point and determining a second representative RGB color space spectrum from the second sampling point; and displaying an overlay of the final edge-perimeter superimposed on the digital wound image.

Still further, another aspect of the present disclosure is directed to a computer-implemented method for detecting an edge-perimeter of a wound from a digital wound image. The method may comprise: receiving one or more sampling lines from a user, wherein the one or more sampling lines follow a shape of the wound on the digital wound image; determining one or more representative color space spectra for the one or more sampling lines based on respective sampling line's multiplicity of points via multi-point sampling of pixels on each of the one or more sampling lines; determining a final edge-perimeter of the wound by determining and combining one or more edge-perimeters for the one or more representative color space spectra; and displaying an overlay of the final edge-perimeter superimposed on the digital wound image.

Yet another aspect of the present disclosure is direct to a computer-implemented system for detecting an edge-perimeter of a wound from a digital wound image. The system may comprise: at least one non-transitory computer-readable medium configured to store instructions; and at least one processor configured to execute the instructions to perform operations. The operations may comprise: receiving one or more sampling lines from a user, wherein the one or more sampling lines follow a shape of the wound on the digital wound image; determining one or more representative color space spectra for the one or more sampling lines based on respective sampling line's multiplicity of points via multi-point sampling of pixels on each of the one or more sampling lines; determining a final edge-perimeter of the wound by determining and combining one or more edge-perimeters for the one or more representative color space spectra; and displaying an overlay of the final edge-perimeter superimposed on the digital wound image.

Other systems, methods, and computer-readable media are also discussed herein.

### BRIEF DESCRIPTION OF THE DRAWINGS

FIG. 1 is a schematic diagram illustrating an exemplary embodiment of a networked environment comprising computerized systems for capturing, uploading, and processing digital wound images, consistent with disclosed embodiments.

FIG. 2 is an exemplary user interface flow of a wound management software application for capturing, uploading, and processing digital wound images, consistent with disclosed embodiments.

FIG. 3 is an exemplary user interface for managing digital wound images, consistent with disclosed embodiments.

FIG. 4 is an exemplary flowchart of a point pixel sampling technique for detecting an edge-perimeter from a digital wound image, consistent with disclosed embodiments.

FIG. 5 is a set of pictographic representations of edge-perimeters detected using the point pixel sampling technique, consistent with disclosed embodiments.

FIG. 6 is an exemplary flowchart of a line pixel sampling technique for detecting an edge-perimeter from a digital wound image, consistent with disclosed embodiments.

FIG. 7 is a set of pictographic representations of edge-perimeters detected using the line pixel sampling technique, consistent with disclosed embodiments.

FIG. 8 is an exemplary user interface for capturing a digital wound image from a patient, consistent with disclosed embodiments.

FIGS. 9A-9C are exemplary user interfaces for characterizing digital wound images, consistent with disclosed embodiments.

FIG. 10 is a set of simplified representations for calculating an area of a wound based on a detected edge-perimeter, consistent with disclosed embodiments.

### DETAILED DESCRIPTION

The following detailed description refers to the accompanying drawings. Wherever possible, the same reference numbers are used in the drawings and the following description to refer to the same or similar parts. While several illustrative embodiments are described herein, modifications, adaptations and other implementations are possible. For example, substitutions, additions, or modifications may be made to the components and steps illustrated in the drawings, and the illustrative methods described herein may be modified by substituting, reordering, removing, or adding steps to the disclosed methods. Accordingly, the following detailed description is not limited to the disclosed embodiments and examples. Instead, the proper scope of the invention is defined by the appended claims.

Embodiments of the present disclosure are directed to systems and methods for enabling a user to make or generate, at any chosen, instant point in time, certain singular or progressive inspections, measurements, evaluations, determinations, calculations, recordings, and storage of a particular wound's instant state or stage of healing, presence of visible tissues, edge-perimeter, dimensions, and area calculations (“outputs or utilities”). The disclosed embodiments may utilize a digital image capture device, uploaded digital images of the particular wound as captured by the digital image capture device at the chosen point in time, and/or a remote server having designated software applications for provision of the utilities or generation of the outputs.

The disclosed embodiments may receive and use digital images of wounds for purposes of displaying, measuring, evaluating, determining, calculating, and/or otherwise using pertinent wound-information taken or derived from the digital images. For example, the disclosed embodiments may provide input for initially and progressively diagnosing wound conditions; prescribing treatment strategies; monitoring and gauging treatment efficacies; determining a state or rate of healing; keeping records on the wound's course of healing; and/or making other uses of the digital images directed or related to care of patients' wounds. Further, the inputs from the disclosed embodiments may be used to track types of treatments used, types of dressings used, and/or patient outcomes on both chronic and non-chronic wounds. In a preferred embodiment, the disclosed embodiments may be configured for use with chronic wounds.

More particularly, disclosed embodiments provides methods and systems using remotely or locally captured digital images of a wound and user-initiated applications of computer processing for delivery of objective, reliable, progressively consistent measurements of the wound's changing edge-perimeter, dimensions, and area during the wound's healing process. The disclosed embodiments enable assessment of the wound's state and course of healing through or by means of non-contact acquisition; minimal user initiation or control; essentially automatic computer-processing; and/or progressively reliable measurements, determinations, and outputs taken from a digital wound image or a progressive series of digital wound images.

Further, the disclosed embodiments provide new or improved minimal contact methods or systems for wound measurement that use digital image capture devices equipped with commonly employed RGB pixel color spectra, along with computer processing or cloud computing by remotely maintained computer servers with necessary software services. The software provides executable programs built with suitable algorithms and outputs. The new or improved methods and systems also consistently provide or allow for an essentially automatic identification and digital display of a depicted healing wound's tissues and its edge-perimeter or boundary on a computer screen.

Except for measurement-calibration, such new or improved methods and systems require minimal user-input beyond an initiation step used to identify wound tissue. And other than a user command for such initiation of processing for image identification and dimension analytics, the new or improved methods and systems limit, simplify, or minimize the user's involvement thereafter in capturing, uploading, and processing a digital wound image of interest presented on the computer screen, while concomitantly enabling the user nevertheless to more systematically, accurately, reliably, consistently, and cost effectively identify, measure, track, and record progressive changes to the healing wound's dimensions, for example, the wound's shape, size, edge-perimeter or boundary, and/or total area.

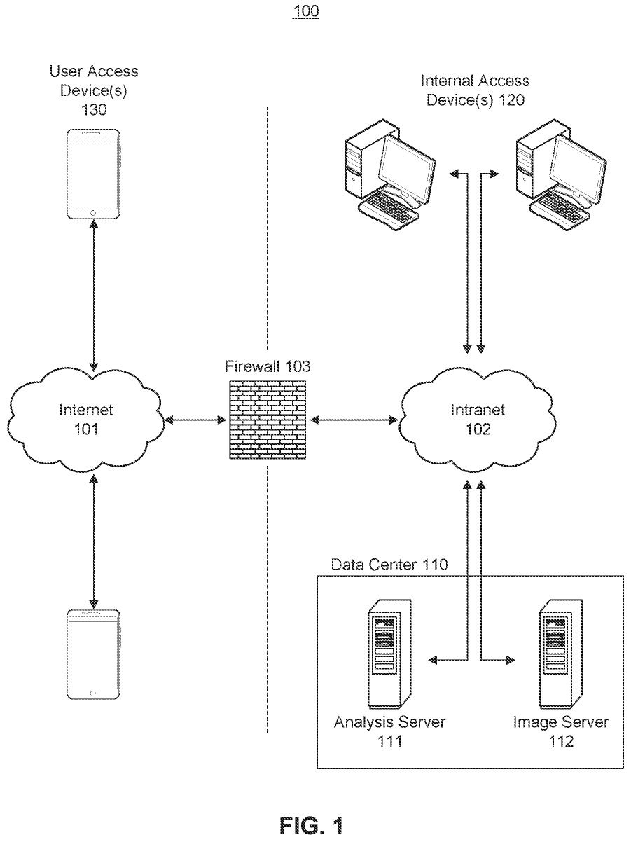

FIG. 1 is a schematic diagram illustrating an exemplary embodiment of a networked environment 100 comprising computerized systems for capturing, uploading, and processing digital wound images. Networked environment 100 may comprise a variety of computerized systems, each of which may be connected to one another via one or more network connections such as the Internet 101 and an intranet 102, separated and protected by a firewall 103. In some embodiments, networked environment 100 comprises a data center 110, internal access device(s) 120, and user access device(s) 130. Each of the systems depicted in FIG. 1 may represent a group of systems, individual systems in a network of systems, an individual computing device, functional units or modules inside of a system, or any combination thereof. In some embodiments, each of the elements may communicate with each other via one or more public or private network connections such as the Internet 101 and intranet 102, which include a WAN (Wide-Area Network), a MAN (Metropolitan-Area Network), a wireless network compliant with the IEEE 802.11 Standards, a wired network, or the like.

Data center 110 may be the central storage and analysis system that comprises an analysis server 111 and an image server 112. Analysis server 111 and image server 112 may each comprise a single computer server or may each be configured as a distributed computer system including multiple computers that interoperate to perform one or more of the processes and functionalities associated with the disclosed embodiments.

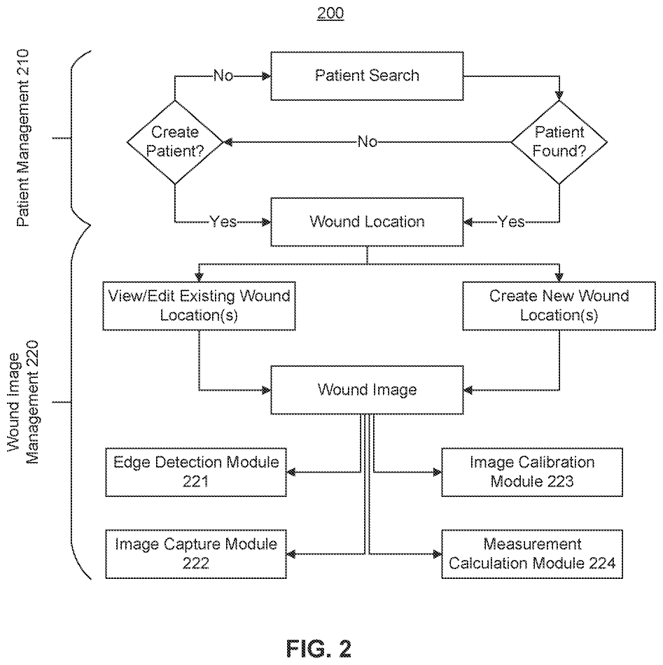

In some embodiments, analysis server 111 is configured to include an image characterization module that can process digital images stored in image server 112. Functions of the image characterization module are discussed below with respect to FIG. 2. The image characterization module may be configured to run within analysis server 111 and be accessible by user access devices 130 in a Software-as-a-Service configuration.

Analysis server 111 comprises a processor and a memory. The processor may comprise one or more known processing devices, such as a microprocessor from any of the processor families manufactured by Intel or AMD. The microprocessor may also include ARM based processors. The processor may constitute a single core or multiple core processor that executes parallel processes simultaneously. For example, the processor may use logical processors to simultaneously execute and control multiple processes. The processor may implement virtual machine technologies or other known technologies to provide the ability to execute, control, run, manipulate, store, etc. multiple software processes, applications, programs, etc. In another example, the processor may include a multiple-core processor arrangement configured to provide parallel processing functionalities to allow analysis server 111 to execute multiple processes simultaneously. One of ordinary skill in the art would understand that other types of processor arrangements could be implemented that provide for the capabilities disclosed herein.

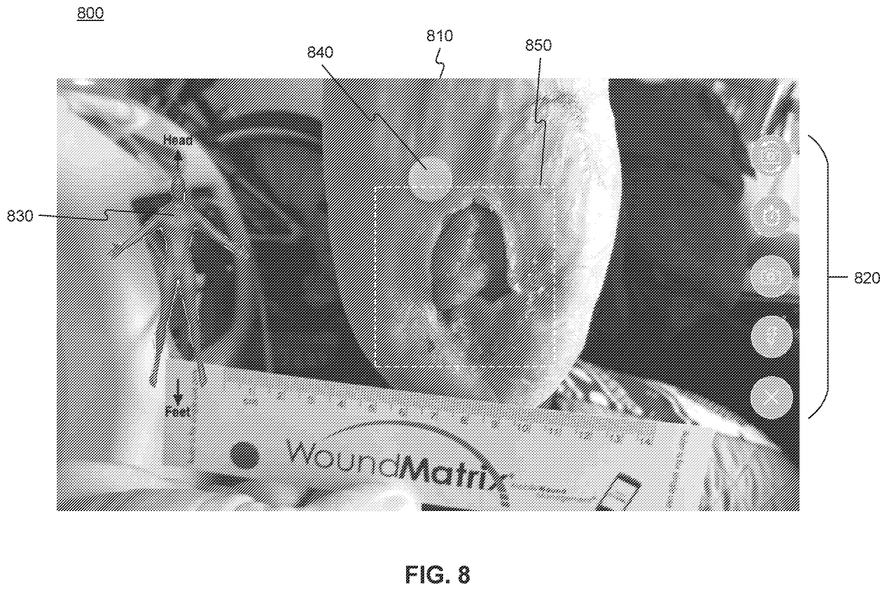

Referring again to data center 110, image server 112 is configured to store and manage digital images of wounds. The digital images may be captured and/or uploaded to image server 112 using user access devices 130. The process for such capture is described below with respect to FIG. 8. In some embodiments, previously captured digital images may be uploaded for analysis as well.

Image server 112 includes computing components (e.g., database management system, database server, etc.) configured to receive and process requests for data stored in memory devices of image server 112 and to provide data from the server. Image server 112 may include NoSQL databases such as HBase, MongoDB™ or Cassandra™. Alternatively, image server 112 may include relational databases such as Oracle, MySQL and Microsoft SQL Server. In some embodiments, image server 112 may take the form of servers, general purpose computers, mainframe computers, or any combination of these components.

In some embodiments, analysis server 111 and image server 112 are connected via intranet 102 to each other and to internal access devices 120. In other embodiments, each element may be located remotely and be connected via secure network connections (not shown) over Internet 101. In other embodiments, user access devices 130 may also be connected to data center 110 via intranet 102. Regardless of the connection configuration, internal access devices 120 may be implemented as computer systems configured to set up and manage data center 110, as well as analysis server 111 and image server 112 therein. For example, internal access devices 120 may be configured to provide system administrators an ability to configure and manage the image characterization module in analysis server 111 or the digital images stored in image server 112.

In some embodiments, user access devices 130 may also be implemented as a computer system like internal access devices 120 and take the form of personal computing devices such as a desktop, a laptop or notebook computer, a smartphone, a tablet, a multifunctional watch, a pair of multifunctional glasses, or any stationary, mobile, or wearable device with computing ability, or any combination of these computing devices and/or affiliated components. User access devices 130, however, may be authorized to access user-facing capabilities of data center 110 (to be explained below), whereas internal access devices 120 may be authorized to access all aspects of data center 110, including but not limited to all features accessible by user access devices 130, algorithms of the image characterization module, administrative modules, and/or reporting modules. In some embodiments, user access devices 130 may be the primary or the sole avenue through which an authorized user may use capabilities of the disclosed embodiments for capturing and processing digital wound images.

In some embodiments, user access devices 130 may comprise one or more display devices (not shown) and one or more input devices (not shown). The display devices may include, for example, a liquid crystal display (LCD), a light emitting diode screen (LED), an organic light emitting diode screen (OLED), a touch screen, or other known display devices. The display devices may be configured to display various user interfaces for interacting with the image characterization module and digital wound images. The input devices may include a keyboard, a mouse-type device, a gesture sensor, an action sensor, a physical button, switch, microphone, touchscreen panel, stylus, etc., to be manipulated by a user to input information or commands on user access device 130.

While each element of networked environment 100 is described above as a discrete system, an alternative embodiment may be possible or desirous under certain circumstances. For example, the functionalities of each element (i.e., data center 110, internal access device 120, and user access device 130) or a subset thereof may be implemented and entirely contained within a single user access device 130. In such embodiments, the single user access device 130 may be configured to capture digital wound images, manage previously captured images, and/or process the images using the image characterization module to determine edge-perimeter of the wounds and/or corresponding parameters. Such embodiment may be advantageous, for example, where data privacy is of concern or the functions of the disclosed embodiments are necessary in the absence of an internet connection.

FIG. 2 is an exemplary user interface flow 200 of a wound management software application for capturing, uploading, and processing digital wound images. In some embodiments, the wound management application may be installed in user access devices 130, or be installed in analysis server 111 and be accessible through user access devices 130. While not shown in user interface flow 200, the wound management application may also include functions for authenticating users, using the wound management application offline, and/or capturing new digital wound images offline for future upload to image server 112.

Generally, main functions that the wound management application provides fall into two groups: patient management 210 and wound image management 220. Patient management 210, in some embodiments, includes functionalities to organize patients by patient identifiers such as a patient ID, name, social security, or the like. The wound management application may allow a user, via user access device 130, to access patient record of a particular patient or create a new patient record. In some embodiments, each patient record includes the corresponding patient's health information such as the patient's name, age, gender, medical history, current health condition, or the like. Furthermore, the wound management software may allow a user to search for a particular patient by any of the information included in the patient record.

Wound image management 220, on the other hand, includes functionalities to organize digital wound images for each patient in the patient record. In some embodiments, wound image management 220 may store multiple digital wound images for each patient record, where one patient record comprises one or more wound locations and each wound location comprises one or more digital wound images. Wound image management 220 may also provide, to users, functions to create new wound locations or view or edit existing wound locations.

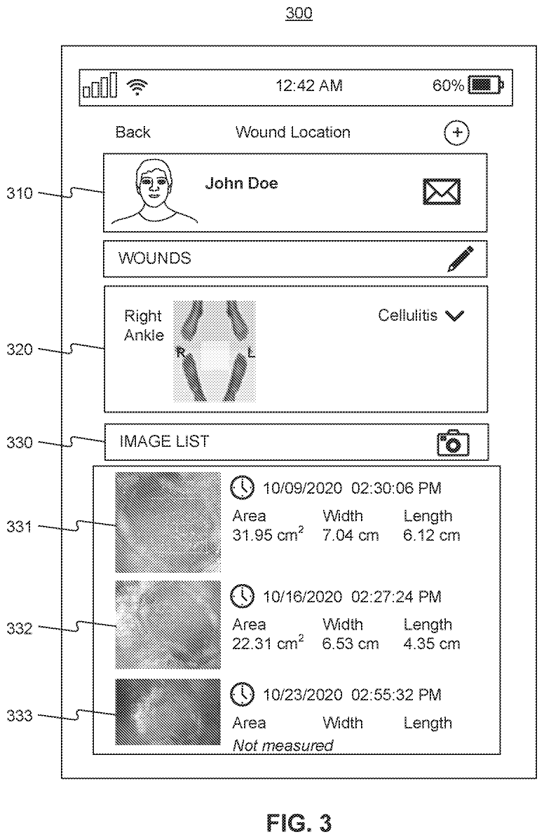

FIG. 3 is an exemplary user interface 300 of the wound management application, showing an exemplary patient record 310; an exemplary wound location 320 among one or more wound locations; and an exemplary image list 330. As shown, image list 330 includes wound images 331-333 with timestamps of when the corresponding wound image was captured and measurements of the wound, such as area, width, and length. All information depicted in FIG. 3 is intended to serve as an example only, and no limiting effect is intended by way of the layout, labels, images, numbers, or data.

In some embodiments, patient records are stored with the wound measurements (e.g., width, length, and area) determined from digital wound images corresponding to respective patient record. These wound measurements, along with the corresponding digital wound images and their timestamps, may allow the user to track progress of a wound over time. Comparison of the wound measurements over time may be an important metric for clinicians to assess whether a particular wound is healing or worsening and identify any medical attention that may be necessary.

Referring back to FIG. 2, wound image management 220 is further configured to detect an edge-perimeter from digital wound images, capture new digital wound images, calibrate the image characterization module's tools for digital wound image measurements, and/or calculate measurements for the detected wounds, using an edge detection module 221, an image capture module 222, an image calibration module 223, and/or measurement calculation module 224, respectively. In some embodiments, at least one of edge detection module 221, image capture module 222, image calibration module 223, and dimension calculation module 224 may be submodules of the image characterization module discussed above. Functions of each module are described in further detail below.

In some embodiments, edge detection module 221 is configured to detect an edge-perimeter of a wound depicted in a digital wound image. As used herein, the edge-perimeter refers to an outermost boundary of the wound, where tissues located inside of the edge-perimeter are considered wound tissues and tissues outside are considered non-wound tissues (i.e., healthy tissues).

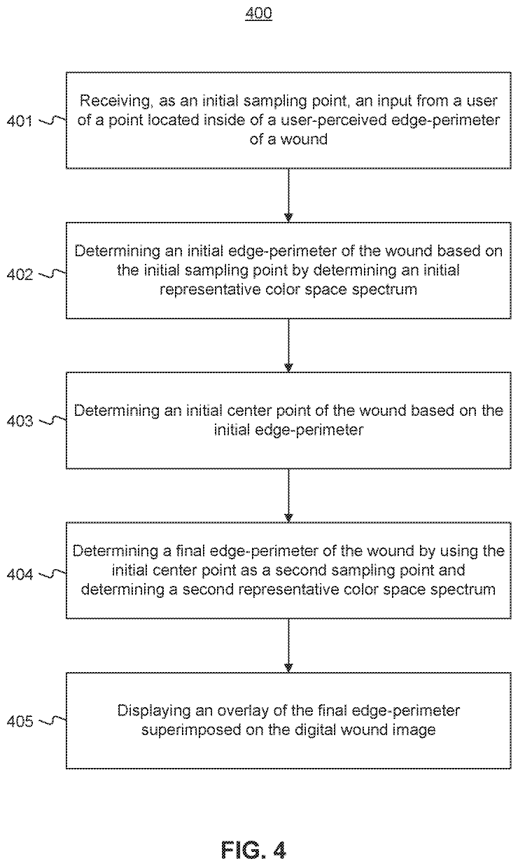

As described in more detail with respect to FIGS. 4-7, edge detection module 221 may employ one or more pixel sampling techniques. Pixel sampling techniques rely on representative color space spectra determined from one or more sampling points and/or sampling lines from digital wound images. In some embodiments, representative color space spectra may be constructed within a particular color space such as RGB or hyperspectral color spaces. In further embodiments, the color space may be limited to RGB and not include colors beyond visible spectrum. Within such color spaces, a representative color-space spectrum may include unique colors of the pixels found within one or more sampling points and/or sampling lines depending on the particular pixel sampling technique employed.

Based on this color spectrum, edge detection module 221 is configured to detect an edge-perimeter using any one or a combination of techniques discussed below.

FIG. 4 is an exemplary flowchart of a point pixel sampling technique 400 for detecting an edge-perimeter from a digital wound image.

At step 401, edge detection module 221 receives an input from a user, via user access device 130, of a single point located inside of a user-perceived edge-perimeter of a wound depicted in a digital wound image displayed on user access device 130. The single point may be inputted via any input device on user access device 130 configured to select a particular location on the displayed digital wound image, such as a mouse, touchscreen, stylus, or the like.

As used herein, the term user-perceived edge-perimeter refers to the edge-perimeter of a wound as the user recognizes it from the digital wound image. In some embodiments, edge detection module 221 may be configured to manipulate displayed color of the digital wound image to assist the user in perceiving the edge-perimeter. The displayed color may be, for example, inverted, contrast-enhanced, brightness-adjusted, or otherwise adjusted using commonly known image processing techniques.

The inputted point may serve as an initial sampling point for subsequent steps. The inputted point may be a particular pixel selected by the user or a group of pixels corresponding to a location on the digital wound image selected by the user. The initial sampling point may be determined from the inputted point as a predetermined area of pixels surrounding the inputted point. In some embodiments, the initial sampling point may have a circular area sized between 1800 to 2200 square pixels. In preferred embodiments, the circular area may be sized between 1800 to 2000 square pixels, and in a most preferred embodiment, the circular area's size is 2000 square pixels.

At step 402, edge detection module 221 determines an initial edge-perimeter of the wound by determining an initial representative color space spectrum from the initial sampling points. In some embodiments, the initial representative color space spectrum is constructed to include every color of the pixels found in the initial sampling points (e.g., every discrete color found among 2000 pixels of an initial sampling point). Once the initial representative color space spectrum is determined, edge detection module 221 begins by analyzing a first set of pixels corresponding to the initial sampling point for conformity with the initial representative color space spectrum. As used herein, colors that “conform” to a color space spectrum refer to those that fall within the color space spectrum (i.e., the color has similar or identical RGB values as a particular color found in the color space spectrum), and colors that “outlie” or “differ” from a color space spectrum refer to those that fall outside of the color space spectrum. In some embodiments, colors that conform to a color space spectrum may include those that fall outside of the color space spectrum by a predetermined threshold.

After analyzing the pixels that correspond to the initial sampling point, edge detection module 221 starts analyzing surrounding pixels, working away progressively outward from the initial sampling point and towards edges of the digital wound image. During this process, edge detection module 221 identifies pixels with a color that conforms with the initial representative color space spectrum. The identified pixels, along with the first set of pixels corresponding to the initial sampling point, correspond to wound tissues.

On the other hand, edge detection module 221 may also identify a second set of pixels that has an outlying color that differs from the initial representative color space spectrum. The second set of pixels correspond to the edge-perimeter of the wound. Edge detection module 221 may stop identifying pixels when the collection of the second set of pixels with outlying colors (i.e., corresponding to the edge-perimeter) forms a closed loop bounded by the collection of the pixels. Edge detection module 221 may use algorithms such as Sobel, Prewitt, Roberts, Canny, and Laplacian or Gaussian methods in order to identify the pixels.

At step 403, edge detection module 221 determines an initial center point of the wound based on the initial edge-perimeter. In some embodiments, determining the initial center point comprises determining a first pair of coordinates of pixels with the highest and the lowest horizontal coordinates and a second pair of coordinates of pixels with the highest and the lowest vertical coordinates. An average of the horizontal coordinates of the first pair of coordinates may be the horizontal coordinate of the initial center point, and an average of the vertical coordinates of the second pair may be the vertical coordinate.

At step 404, edge detection module 221 determines a final edge-perimeter of the wound by using the initial center point as a second sampling point and determining a second representative color space spectrum based on the second sampling point. In some embodiments, step 404 may be substantially similar to step 402 except that different sampling points are used.

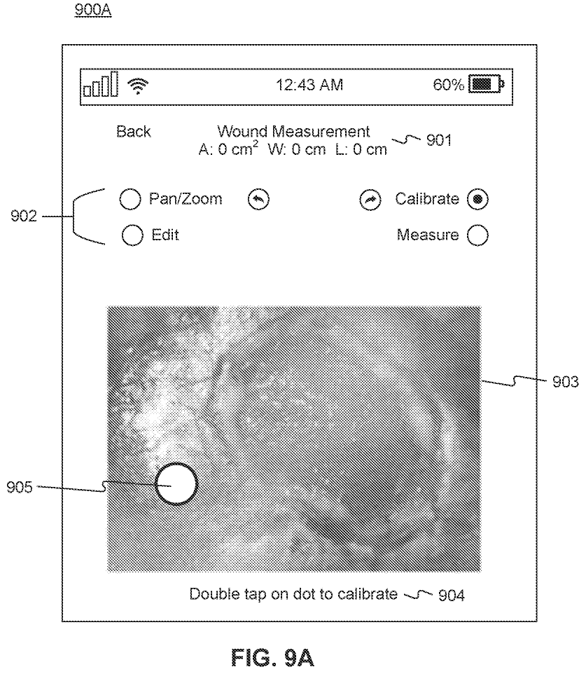

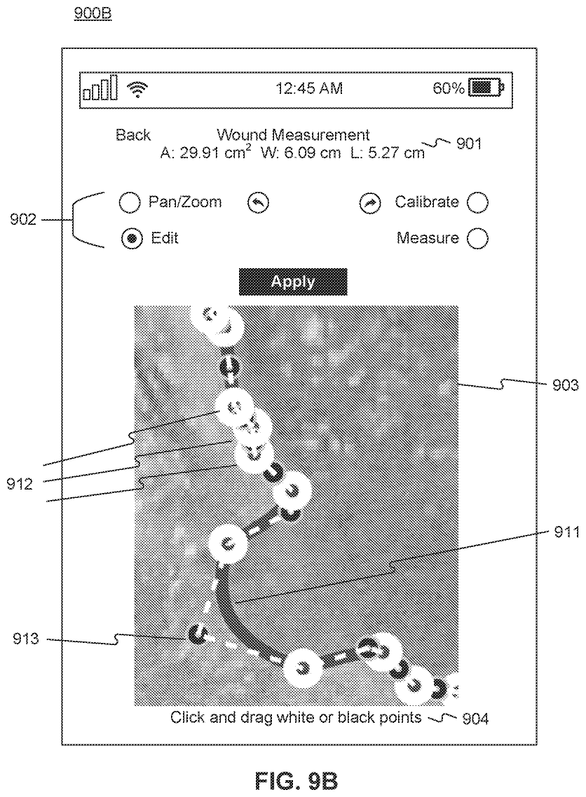

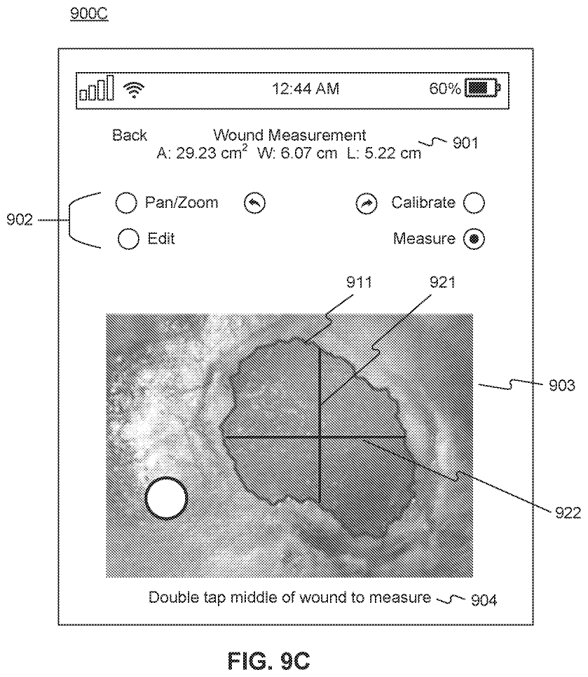

At step 405, edge detection module 221 displays an overlay of the final edge-perimeter superimposed on the digital wound image. The overlay may be displayed in a manner that makes the edge-perimeter easily distinguishable from the rest of the digital wound image. For example, the overlay may be brightly colored, highlighted, displayed with a color contrasting with the second representative color space spectrum, or any combination thereof. Additionally, or alternatively, the overlay may be semi-transparent, so as to not obstruct view of the underlying wound image. In some embodiments, edge detection module 221 may also provide the user an ability to fine tune the edge-perimeter as described below with respect to FIG. 9B.

The point pixel sampling technique 400 of receiving a first sampling point and determining the final edge-perimeter in an iterative process may be referred as double point pixel sampling technique. In some embodiments, the final edge-perimeter determined using the double point pixel sampling technique may be more accurate and yield a final center point that is truer than the initial center point.

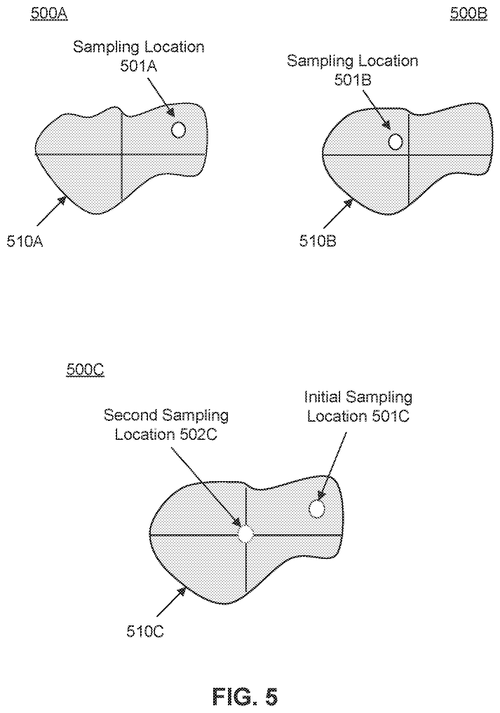

FIG. 5 is a set of pictographic representations of detected edge-perimeters using point pixel sampling technique 400. Representation 500A and representation 500B depict exemplary edge-parameters determined without the iterative process of determining the final edge-parameter after determining a second sampling point. Representation 500C depicts an exemplary edge-parameter determined using the double point pixel sampling technique. Representations 500A-500C are assumed to be determined from the same hypothetical digital wound image.

As is apparent from representations 500A and 500B, using two spatially distinct sampling locations 501A and 501B and not going through the process of redetermining the edge-perimeter based on the second sampling point can lead to markedly different edge-perimeters 510A and 510B. Such discrepancy may be caused by different color space spectra determined from the pixels located at sampling locations 501A and 501B. The different color space spectra may be due to different factors such as noise, lighting conditions, orientation of the wound, or the like that can affect how color is captured in a digital image. In such cases, the closer a selected sampling point is to the actual center point of the wound may result in the final edge-perimeter being more accurate and/or lead to a more consistent result. For example, edge-perimeter 510B is a more accurate representation of the wound in the digital wound image in this example.

In contrast, the double point pixel sampling technique may lead to consistent results even where initial sampling location 501C is relatively far away from the actual center point of the wound. For example, edge-perimeters 510B and 510C are similar to each other even though initial sampling location 501C is farther from the actual center point (i.e., the second sampling location 502C or the intersection of the horizontal and vertical lines in representation B 500B) than sampling location 501B.

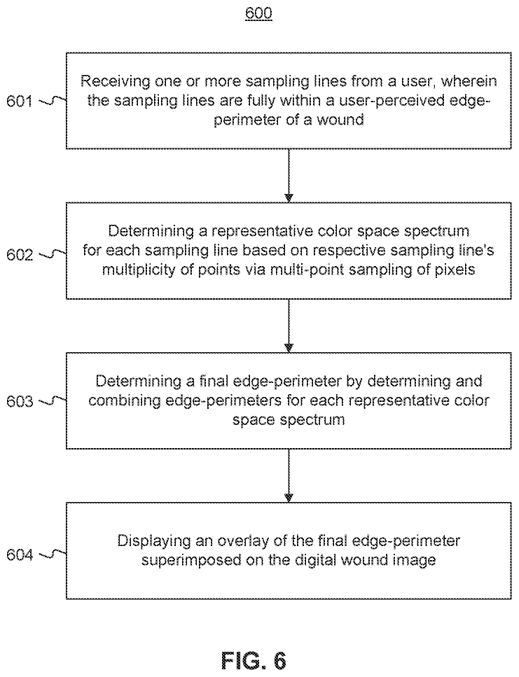

Further considering the techniques edge protection module 221 may employ to detect edge-perimeters, FIG. 6 is an exemplary flowchart of a line pixel sampling technique 600 for detecting an edge-perimeter from a digital wound image.

At step 601, edge detection module 221 receives, from a user via user access device 130, one or more straight or curvilinear sampling lines. The sampling lines may be inputted via any input device on user access device 130 configured to generate a line or a series of points, such as a mouse, touchscreen, stylus, or the like. For example, user access device 130 may comprise a touchscreen and the user may draw one or more lines directly on the wound image displayed on the touchscreen using a finger.

The one or more sampling lines may follow at least a portion of a shape of a wound depicted in a digital wound image displayed on user access device 130. In some embodiments, the one or more sampling lines may lie fully within a user-perceived edge perimeter of the wound, which means that no portion of the sampling lines falls outside of the user-perceived edge-perimeter. Each sampling line may be a lone, single, open-ended line that appears on the wound image as discrete and unbroken.

In further embodiments, a sampling line drawn by the user may, along its length, be entirely, partially, or intermittently: (i) linear, (ii) straight, (iii) non-linear, (iv) other than entirely linear or straight, (v) angled, multi-angled, or non-angled, (vi) curvilinear, multi-curvilinear or non-curvilinear, (vii) open-ended at each end, but along its length self-intersecting or non-self-intersecting in any of its other configurations, or (viii) be comprised of any random combination of such configurations or appearances.

For any given wound image displaying a more complex, differentiated appearance, configuration, or shape, the user may optionally input an additional sampling line within the user-perceived edge-perimeter as an alternative or additional sampling line. If desired, the user may also optionally input an additional sampling line. For any given wound image displaying an even more complex, differentiated appearance, configuration, or shape, the user may input even more additional sampling lines within the user-perceived edge-perimeter as further alternative or additional sampling lines.

At step 602, edge detection module 221 determines a representative color space spectrum for each of the one or more sampling lines' multiplicity of points via multi-point sampling of pixels. The multiplicity of points may correspond to a series of points that comprise each sampling line, where each point may include a set of pixels found within a predetermined radius from a center. For example, edge detection module 221 may decompose each sampling line into a series of mid-point pixels that run through the middle of a sampling line. The multiplicity of points may include the set of pixels found within, e.g., 10 pixels of the mid-point pixels, and the representative color-space spectrum may include every discrete color found within the multiplicity of points. In other embodiments, edge detection module may take multiple samples along each sampling line and set the multiplicity of points as the set of pixels found within, e.g., 10 pixels of the samples.

Each such representative color space spectrum determined from the multiplicity of points on a sampling line has been found to be more accurate than a representative color space spectrum determined from a single point in the double point pixel sampling technique discussed above. This may be due to the more expansive pool of pixels that are used to construct the representative color space spectrum, where each sampling line comprises of a multiplicity of sampling points.

In further embodiments, edge detection module 221 may determine a representative color space spectrum for each discrete sampling line and combine the resulting spectra to determine a composite, overarching representative color space spectrum.

At step 603, edge detection module 221 determines a final edge-perimeter of the wound based on the representative color space spectra or the composite representative color space spectrum. In some embodiments, this step may be similar to steps 402 or 404 of FIG. 4, except that edge detection module 221 may start searching from the multiplicity of points on the one or more sampling lines rather than a single point as was the case in steps 402 and 404.

In some embodiments, edge detection module 221 may determine an edge-perimeter for each representative color space spectrum determined for each sampling line. This process may comprise determining a line sampling point for each sampling line, and working away from respective line sampling point to identify pixels with colors conforming to the corresponding representative color space spectrum of the corresponding sampling line. As was the case in steps 402 and 404, pixels with colors that differ from the corresponding representative color space spectrum may indicate that the pixels correspond to an edge-perimeter of the wound. The identifying process for each sampling line may end when a set of non-conforming pixels associated with non-wound tissue (that is, pixels having colors that differ from the corresponding representative color space spectrum associated with wound tissue) form a continuous, closed loop within the wound image. As a final step, edge detection module 221 may determine the final edge-perimeter by combining the edge-perimeters determined for the representative color space spectra.

In another embodiment, edge detection module 221 may determine the final edge-perimeter using the composite representative color space spectrum. Here, edge detection module 221 may start from a point on a sampling line and work away progressively outward from the point and towards edges of the digital wound image. Edge detection module 221 may repeat this process for additional points on the sampling line and for even more points on the other sampling lines until the set of non-conforming pixels form a continuous, closed loop as described above.

At step 604, edge detection module 221 displays an overlay of the edge-perimeter superimposed on the digital wound image. As was the case in step 405 of FIG. 4, the overlay may be displayed in a manner that makes the edge-perimeter easily distinguishable from the rest of the digital wound image and/or displayed in a way that does not obstruct the view of the underlying wound image.

In some embodiments, edge detection module 221 performs this process of receiving an initial sampling line and optionally one or more additional sampling lines (step 601), determining a representative color space spectrum for the initial sampling line or a collective set comprised of the initial and additionally inputted sampling lines (step 602), determining a work-in-progress edge-perimeter for the representative color space spectrum of only the initial sampling line or the work-in-progress spectrum of the collective set, and finally determining the edge-perimeter iteratively as either one derived from only the initial sampling line or one derived from the work-in-progress edge-perimeter derived from the collective set of sampling lines inputted by the user (“final edge-perimeter”) (step 603). In such embodiments, each edge-perimeter determination, derived either from such initial or collective set of single sampling lines inputted (initially by itself, or additionally as a collective work-in-progress), is combined cumulatively for determining a final edge-perimeter. This allows edge detection module 221 to display a work-in-progress edge perimeter and the final edge-perimeter as an overlay on the digital wound image and update the overlay each time an additional sampling line is inputted and a corresponding edge-perimeter is added to the final edge-perimeter. This may serve to provide the user a real time feedback from the sampling lines he or she input, thereby improving the accuracy of the final edge-perimeter.

In further embodiments, when the user's additional sampling line, once incorporated cumulatively to the final edge-perimeter, causes the final edge-perimeter to expand, fall, or protrude beyond the user-perceived edge-perimeter, the user may optionally make further adjustments by withdrawing or removing any of the previously drawn sampling lines. Withdrawing a particular sampling line may prompt edge detection module 221 to remove the particular sampling line and redetermine the edge-perimeter based on remaining sampling lines. The user may add new sampling lines again to continue determining the edge-perimeter. In some embodiments, the adjustments by the user may include: (i) withdrawing any previously-drawn additional sampling line; (ii) withdrawing successively one or more previously-drawn additional sampling lines and replacing the withdrawn sampling lines with one or more differently-drawn sampling lines at or about the same location; or (iii) withdrawing successively one or more last-drawn additional sampling lines and replacing the withdrawn sampling lines with one or more differently-drawn sampling lines at or about different locations.

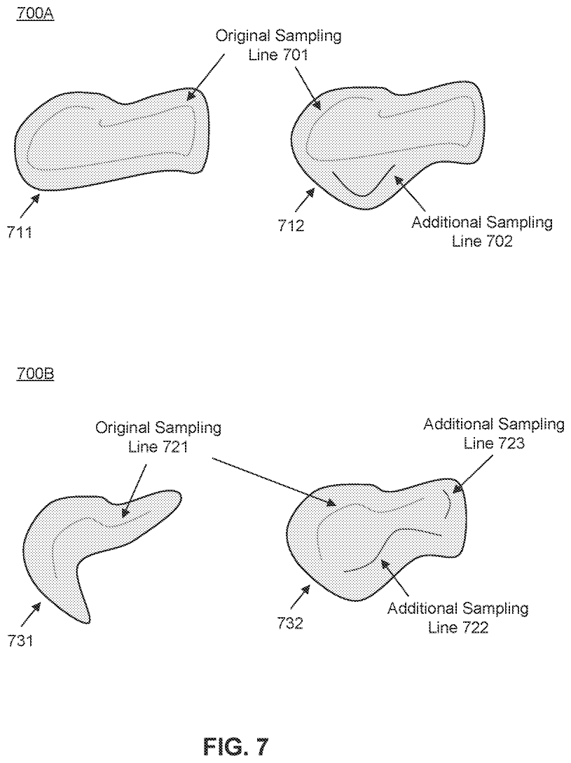

FIG. 7 is a set of pictographic representations of detected edge-perimeters using line pixel sampling technique 600. A top pair of representations 700A and a bottom pair of representations 700B depict edge-perimeters of an exemplary wound based on the same hypothetical digital wound image.

Referring to the top pair of representations 700A, an exemplary edge-perimeter 711 is determined based on an original sampling line 701. An updated exemplary edge-perimeter 712 is determined based on the existing edge-perimeter 711, updated with an edge-perimeter determined from an additional sampling line 702. Referring to the bottom pair of representations 700B, another exemplary edge-perimeter 731 is determined based on an original sampling line 721. An updated exemplary edge-perimeter 732 is determined based on the existing edge-perimeter 731, updated with edge-perimeters determined from additional sampling lines 722 and 723.

As shown by the similarity between edge-perimeters 712 and 732, there may be many different combinations of sampling lines that can result in substantially similar edge-perimeters. This advantage allows for more human variability in wound image analysis, where even a relatively low skilled user may accurately determine edge-perimeters of wounds.

Referring back to the submodules of the image characterization module, FIG. 8 is an exemplary user interface 800 for capturing a digital wound image from a patient using image capture module 222.

In some embodiments, a user may capture digital wound images using a digital image capture device of user access device 130 and upload them to image server 112. The digital image capture device may be a digital camera (or an analog camera capable of producing an analog image capable of digital conversion), mobile telephone capable of capturing or taking digital images, or any other electronic device configured to capture an image in a digital or analog format. In general, to expedite capture and use of the given wound image, the digital image capture device may be in the form of a digital camera or a smartphone equipped with a digital camera. Without limitation, examples of such digital image capture devices include the following: an Apple iPad or iPhone, and an Android tablet or mobile phone. Additionally or alternatively, the digital image capture device may include imaging sensors capable of capturing wavelengths beyond the visible spectrum, such as a hyperspectral sensor, infrared sensor, or the like.

In some embodiments, image capture module 222 may be configured to scale or crop the captured images to optimize the images for uploading and/or processing. Scaling the captured images may comprise reducing the resolution of the images. While a higher resolution image may be desirous for accurate wound measurements, excessively high resolution images may increase burden on the image characterization module. For example, the increased number of pixels in higher resolution images may contribute to longer processing time to detect edge-perimeter or measure wound dimensions. Scaling down the captured images to a reasonable size or resolution while maintaining the image quality or clearness may enhance the performance of the image characterization module, which may speed up the processing time and/or enable real time processing of captured images. Cropping the captured image to contain a full view of a wound and little extra surrounding tissue may also achieve a similar effect by reducing the number of pixels that the image characterization module must process.

In some embodiments, user interface 800 comprises an image pane 810, camera control buttons 820, a navigational avatar 830, a calibration dot 840, and a bounding box 850. Image pane 810 may be configured to display a current view of the digital image capture device, where the image appearing in image pane 810 is representative of the resulting digital image. Camera control buttons 820 may comprise various user interface elements for configuring parameters of the digital image capture device, such as for selecting a lens, setting a timer, capturing an image, controlling flash, or the like. Navigational avatar 830 may serve to identify a location of the wound by using an avatar of a human body as a reference model and asking the user to specify a location of the wound on a corresponding location on navigational avatar 830. To further improve the accuracy of the wound position on the body, the avatar can be rotated so that the user can specify the position of the wound in a representative three-dimensional space.

Calibration dot 840, as is described below with respect to FIG. 9A, is a token or an object of a known dimension that is placed next to the wound for calibration. Bounding box 850 is a user interface element displayed in image pane 810 to represent an area of the wound. In some embodiments, image capture module 222 may instruct the user to adjust the position of the digital image capture device so that the wound is enclosed entirely within bounding box 850. Additionally or alternatively, user interface 800 may provide further user interface elements (not shown) to control the size or shape of bounding box 850.

FIGS. 9A-9C are exemplary user interfaces for characterizing digital wound images with the image characterization module. More specifically, FIG. 9A is an exemplary user interface 900A for calibrating a digital wound image using image calibration module 223; FIG. 9B is an exemplary user interface 900B for editing an edge-perimeter using edge detection module 221; and FIG. 9C is an exemplary user interface 900C for measuring different parameters of a wound using measurement calculation module 224. User interfaces 900A-900C may be configured to be displayed on user access device 130.

Referring to FIG. 9A, user interface 900A includes a measurement display pane 901, a control pane 902, a wound image pane 903, and an instruction pane 904. As depicted in FIG. 9A, measurement display pane 901 may be empty, indicating that the particular wound image displayed on wound image pane 903 has not been measured yet. Measurement display pane 901 may display previously obtained measurements if any are available for the particular digital wound image currently displayed in wound image pane 903. Control pane 902 may comprise different user interface elements (e.g., radio button as shown) to indicate the current function of user interface 900A. For example, control pane 902 depicted in user interface 900A shows that “calibrate” mode is active. Instruction pane 904 also corroborates this finding, which is configured to display an appropriate instruction for the chosen mode.

User interface 900A may further include a calibration dot 905, which may be located outside of a wound area in the digital wound image. In a preferred embodiment, calibration dot 905 must be located outside of the wound area in the digital wound image. While it is depicted as a graphical user interface element in FIG. 9A, calibration dot 905 may be a tangible token or an object of a known size (e.g., a circle of about 1 cm in diameter) that is placed next to a wound, as in the case of calibration dot 840 in FIG. 8. In other embodiments, calibration dot 905 may be a graphical user interface element identical in size to calibration dot 840 but displayed in wound image pane 903 instead of calibration dot 840.

Once a user selects (e.g., clicks or taps) calibration dot 905 to begin the calibration process, image calibration module 223 calculates the number of pixels located inside calibration dot 905. Measurement of the size of the area under calibration dot 905 (e.g., 3.14 cm²) compared to the number of pixels inside calibration dot 905 (e.g., 1000 pixels) allows image calibration module 223 to calculate a scale between each pixel of the digital wound image and an actual length represented by the pixel.

Referring to FIG. 9B, user interface 900B is similar to user interface 900A in many aspects such as measurement display pane 901, control pane 902, wound image pane 903, and instruction pane 904. The two user interfaces may differ in key places, however, such as control pane 902 showing that “edit” mode is active and instruction pane 904 displaying another line of instruction for the user. Here, user interface 900B is configured to provide the user an ability to manipulate individual points comprising an edge-perimeter 911.

As discussed above, edge-perimeter 911 is a collection of pixels that form a closed loop as determined using the point pixel sampling technique 400, the line pixel sampling technique 600, or the like. Edge-perimeter 911 may also be represented as a series of data points 912 and curvature points 913, where data points 912 are depicted as white points in image pane 903 and curvature points 913 are depicted as black points. As used herein, data points 912 refer to actual points or pixels that make up edge-perimeter 911. Curvature points 913 refer to a point tethered to two neighboring data points 912 and defining a curvature of an edge. In some embodiments, curvature point 913 may be located at the intersection of lines extending from the two neighboring data points 912 and tangent to the curvature of the edge between the two neighboring data points 912.

In some embodiments, edge detection module 221 allows the user to directly manipulate data points 912 and/or curvature points 913. Manipulation of data points 912 or curvature points 913 may affect the shape of edge-perimeter 911, altering positions of the points comprising edge-perimeter 911 or the curvature of edges. In some embodiments, measurement calculation module 224 may recalculate and update wound measurements displayed in measurement display pane 901 as the points on edge-perimeter 911 are adjusted.

Referring to FIG. 9C, user interface 900C is also similar to user interfaces 900A and 900B in many aspects such as measurement display pane 901, control pane 902, wound image pane 903, and instruction pane 904. User interface 900C may differ from the other two interfaces in key places, such as control pane 902 showing “measure” mode as being active and instruction pane 904 displaying yet another line of instruction for the user.

User interface 900C further includes an overlay of edge-perimeter 911, a horizontal axis 921, and a vertical axis 922 of the wound in the wound image. In some embodiments, measurement calculation module 224 determines the wound's horizontal width and vertical length, measured by horizontal axis 921 and vertical axis 922 intersecting at a center point of edge-perimeter 911. Measurement calculation module 224 determines the position of horizontal axis 921 and vertical axis 922 by first determining the center point as discussed above at step 403 of FIG. 4 and extending horizontal axis 921 and vertical axis 922 from the center point to edge-perimeter 911. In some embodiments, determining the width and length may comprise counting the number of pixels in horizontal axis 921 and vertical axis 922, respectively, and using the scale determined by image calibration module 223 above. Measurement calculation module 224 may then update measurement display pane 901 with the calculated width, length, and area.

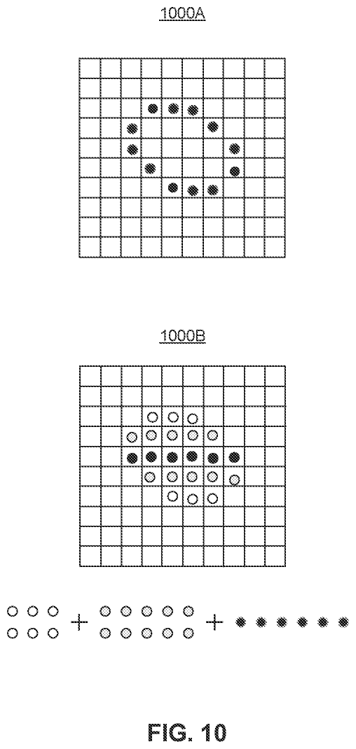

Since edge-perimeter 911 of a wound is rarely smooth like a circle or an oval, measurement calculation module 224 determines the area of the wound by counting the number of pixels that fall within edge-perimeter 911. For example, FIG. 10 is a set of simplified representations 1000A and 1000B for calculating an area of a wound based on a detected edge-perimeter. Representation 1000A depicts an exemplary edge-perimeter, where each square in the grid represents a pixel and each dot represents a pixel corresponding to the edge-perimeter. Representation 1000B depicts a process for counting the number of pixels within the edge-perimeter, where each grid that is located within the edge-perimeter is marked with a dot. Measurement calculation module 224 counts the number of pixels by rearranging the rows of pixels of the same length into rectangles and adding the areas of the rectangles.

While the present disclosure has been shown and described with reference to particular embodiments thereof, it will be understood that the present disclosure can be practiced, without modification, in other environments. The foregoing description has been presented for purposes of illustration. It is not exhaustive and is not limited to the precise forms or embodiments disclosed. Modifications and adaptations will be apparent to those skilled in the art from consideration of the specification and practice of the disclosed embodiments. Additionally, although aspects of the disclosed embodiments are described as being stored in memory, one skilled in the art will appreciate that these aspects can also be stored on other types of computer readable media, such as secondary storage devices, for example, hard disks or CD ROM, or other forms of RAM or ROM, USB media, DVD, Blu-ray, or other optical drive media.

Computer programs based on the written description and disclosed methods are within the skill of an experienced developer. Various programs or program modules can be created using any of the techniques known to one skilled in the art or can be designed in connection with existing software. For example, program sections or program modules can be designed in or by means of .Net Framework, .Net Compact Framework (and related languages, such as Visual Basic, C, etc.), Java, C++, Objective-C, HTML, HTML/AJAX combinations, XML, or HTML with included Java applets.

Moreover, while illustrative embodiments have been described herein, the scope of any and all embodiments having equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present specification or during the prosecution of the application. The examples are to be construed as non-exclusive. Furthermore, the steps of the disclosed methods may be modified in any manner, including by reordering steps and/or inserting or deleting steps. It is intended, therefore, that the specification and examples be considered as illustrative only, with a true scope and spirit being indicated by the following claims and their full scope of equivalents.

What is claimed is:

1. A computer-implemented method for detecting an edge-perimeter of a wound from a digital wound image, the method comprising:
receiving, as an initial sampling point, an input from a user of a point on the digital wound image located inside of the wound;
determining an initial edge-perimeter of the wound based on the initial sampling point by determining an initial representative color space spectrum from the initial sampling point;
determining an initial center point of the wound on the digital wound image based on the initial edge-perimeter;
determining a final edge-perimeter of the wound by using the initial center point as a second sampling point and determining a second representative color space spectrum from the second sampling point; and
displaying an overlay of the final edge-perimeter superimposed on the digital wound image.

2. The computer-implemented method of claim 1, wherein the representative color space spectrum is in a Red-Green-Blue color space.

3. The computer-implemented method of claim 1, wherein determining the initial edge-perimeter of the wound based on the initial sampling point comprises:
analyzing a first set of pixels corresponding to the initial sampling point for conformity with the initial representative color space spectrum; and
working away progressively outward from the initial sampling point to identify a second set of pixels having colors different from those found in the initial representative color space spectrum,
wherein the second set of pixels correspond to the initial edge-perimeter.

4. The computer-implemented method of claim 1, furthering comprising:
calibrating the digital wound image using a user interface element of a known size; and
determining a scale between a single pixel of the digital wound image and an actual length represented by the single pixel.

5. The computer-implemented method of claim 4, further comprising:
determining at least one of a length, a width, or an area of the wound based on the scale; and
storing at least one of the determined length, the width, or the area of the wound over time.

6. A computer-implemented system for detecting an edge-perimeter of a wound from a digital wound image, the system comprising:
at least one non-transitory computer-readable medium configured to store instructions; and
at least one processor configured to execute the instructions to perform operations comprising:
receiving, as an initial sampling point, an input from a user of a point on the digital wound image located inside of the wound;
determining an initial edge-perimeter of the wound based on the initial sampling point by determining an initial representative color space spectrum from the initial sampling point;
determining an initial center point of the wound on the digital wound image based on the initial edge-perimeter;
determining a final edge-perimeter of the wound by using the initial center point as a second sampling point and determining a second representative color space spectrum from the second sampling point; and
displaying an overlay of the final edge-perimeter superimposed on the digital wound image.

7. The computer-implemented system of claim 6, wherein the representative color space spectrum is in a Red-Green-Blue color space.

8. The computer-implemented system of claim 6, wherein determining the initial edge-perimeter of the wound based on the initial sampling point comprises:
analyzing a first set of pixels corresponding to the initial sampling point for conformity with the initial representative color space spectrum; and
working away progressively outward from the initial sampling point to identify a second set of pixels having colors different from those found in the initial representative color space spectrum,
wherein the second set of pixels correspond to the initial edge-perimeter.

9. The computer-implemented system of claim 6, wherein the operations further comprise:
calibrating the digital wound image using a user interface element of a known size; and
determining a scale between a single pixel of the digital wound image and an actual length represented by the single pixel.

10. The computer-implemented system of claim 9, wherein the operations further comprise:
determining at least one of a length, a width, or an area of the wound based on the scale; and
storing at least one of the determined length, the width, or the area of the wound over time.

11. A computer-implemented method for detecting an edge-perimeter of a wound from a digital wound image, the method comprising:
receiving one or more sampling lines from a user, wherein the one or more sampling lines follow a shape of the wound on the digital wound image;
determining one or more representative color space spectra for the one or more sampling lines based on respective sampling line's multiplicity of points via multi-point sampling of pixels on each of the one or more sampling lines;
determining a final edge-perimeter of the wound by determining and combining one or more edge-perimeters for the one or more representative color space spectra; and
displaying an overlay of the final edge-perimeter superimposed on the digital wound image.

12. The computer-implemented method of claim 11, wherein the one or more sampling lines are open-ended, discrete, and continuous.

13. The computer-implemented method of claim 11, further comprising:
removing a subset of the one or more sampling lines;
receiving one or more additional sampling lines from the user; and
updating the final edge-perimeter based on a remaining subset of the one or more sampling lines and the one or more additional sampling lines.

14. The computer-implemented method of claim 11, wherein determining the final edge-perimeter occurs iteratively as each of the one or more sampling lines are received.

15. The computer-implemented method of claim 11, further comprising:
calibrating the digital wound image using a user interface element of a known size;
determining a scale between a single pixel of the digital wound image and an actual length represented by the single pixel;
determining at least one of a length, a width, or an area of the wound based on the scale; and
storing at least one of the determined length, the width, or the area of the wound over time.

16. A computer-implemented system for detecting an edge-perimeter of a wound from a digital wound image, the system comprising:
at least one non-transitory computer-readable medium configured to store instructions; and
at least one processor configured to execute the instructions to perform operations comprising:
receiving one or more sampling lines from a user, wherein the one or more sampling lines follow a shape of the wound on the digital wound image;
determining one or more representative color space spectra for the one or more sampling lines based on respective sampling line's multiplicity of points via multi-point sampling of pixels on each of the one or more sampling lines;
determining a final edge-perimeter of the wound by determining and combining one or more edge-perimeters for the one or more representative color space spectra; and
displaying an overlay of the final edge-perimeter superimposed on the digital wound image.

17. The computer-implemented system of claim 16, wherein the one or more sampling lines are open-ended, discrete, and continuous.

18. The computer-implemented system of claim 16, wherein the operations further comprise:
removing a subset of the one or more sampling lines;
receiving one or more additional sampling lines from the user; and
updating the final edge-perimeter based on a remaining subset of the one or more sampling lines and the one or more additional sampling lines.

19. The computer-implemented system of claim 16, wherein determining the final edge-perimeter occurs iteratively as each of the one or more sampling lines are received.

20. The computer-implemented system of claim 16, wherein the operations further comprise:
calibrating the digital wound image using a user interface element of a known size;
determining a scale between a single pixel of the digital wound image and an actual length represented by the single pixel;
determining at least one of a length, a width, or an area of the wound based on the scale; and
storing at least one of the determined length, the width, or the area of the wound over time.
